SEQUENCE LISTING (14 sequences)

SEQ ID NO: 1 - protein, 8 aa, synthetic construct
GGGSGGGS

SEQ ID NO: 2 - protein, 133 aa, synthetic construct [sequence omitted]

SEQ ID NO: 3 - protein, 8 aa, synthetic construct
GFTFSSYG

SEQ ID NO: 4 - protein, 8 aa, synthetic construct
INSNGGST

SEQ ID NO: 5 - protein, 12 aa, synthetic construct
ARLGTGTLGFAY

SEQ ID NO: 6 - protein, 6 aa, synthetic construct
QDVKTA

SEQ ID NO: 7 - skipped

SEQ ID NO: 8 - protein, 9 aa, synthetic construct
QQHYSTPWT

SEQ ID NO: 9 - protein, 590 aa, synthetic construct
QVQLVESGGGVVQPGRSLRLDCKASGFTFSSYGMSWVRQAPGKGLELVATINSNGGSTYYPDSVKGRFTISRDNSKNTLFLQMNSLRAEDTAVYYCARLGTGTLGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 10 - protein, 214 aa, synthetic construct
EIVMTQSPATLSLSPGERATLSCKASQDVKTAVSWYQQKPGQAPRLLIYWASTRATGIPARFSGSGSGTDYTLTISSLEPEDFAVYYCQQHYSTPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 15 bp, synthetic construct
  source 1..15: mol_type = other DNA
ggcggcggcggcagc

SEQ ID NO: 12 - DNA, 399 bp, synthetic construct
  source 1..399: mol_type = other DNA
cagggccaggacaggcacatgatcaggatgaggcagctgatcgacatcgtggaccagctgaagaactacgtgaacgacctggtgcccgagttcctgcccgcccccgaggacgtggagaccaactgcgagtggagcgccttcagctgcttccagaaggcccagctgaagagcgccaacaccggcaacaacgagaggatcatcaacgtgagcatcaagaagctgaagaggaagccccccagcaccaacgccggcaggaggcagaagcacaggctgacctgccccagctgcgacagctacgagaagaagccccccaaggagttcctggagaggttcaagagcctgctgcagaagatgatccaccagcacctgagcagcaggacccacggcagcgaggacagc

SEQ ID NO: 13 - DNA, 1770 bp, synthetic construct
  source 1..1770: mol_type = other DNA
caggtgcagctggtggaatcaggaggaggagtggtgcagccaggaagaagcctgagactggattgcaaggccagcggcttcaccttcagcagctacggaatgtcttgggtccggcaggctccaggaaaaggactggaactggtggccaccatcaacagcaacggcggcagcacctactacccagacagcgtgaagggcaggttcaccatcagccgggacaacagcaagaacaccctgttcctgcagatgaacagcctgagggccgaggataccgccgtgtactattgcgctagactgggcaccggcacactgggatttgcctattggggccagggcacactggtgacagtgtctagcgccagcaccaagggccctagcgtgtttcctctggccccttctagcaagtctacaagcggcggaacagccgctctgggttgcctggtgaaagactacttccccgagccagtgaccgtgtcttggaatagcggagccctgaccagcggagtgcatacctttccagccgtgctgcagagctcaggactgtacagcctgtccagcgtggtgacagtgccttctagcagcctgggcacacagacctacatctgcaacgtgaaccacaagcccagcaacaccaaggtggacaagaaggtggagcccaagagctgcgacaagacccacacttgccctccttgtccagctccagaactgctgggaggaccaagcgtgttcctgttccctcccaagcccaaggacaccctgatgatcagcaggacccccgaagtgacttgcgtggtggtggacgtgtctcacgaggaccccgaggtcaagttcaattggtacgtggacggagtggaggtgcacaacgctaagaccaagcccagggaggagcagtacaacagcacctacagggtggtgtccgtgctgacagtgctgcaccaggattggctgaacggcaaggagtacaagtgcaaggtgtccaacaaggctctgccagcccccatcgagaagaccatcagcaaggctaagggccagcctagagaacctcaggtgtacaccctgcccccttctagagacgagctgaccaagaaccaggtgtccctgacttgcctcgtgaagggcttctaccccagcgatatcgccgtggagtgggaatctaacggccagccagagaacaactacaagaccacccccccagtgctggacagcgacggcagcttcttcctgtacagcaagctgaccgtggacaaaagccgctggcagcagggcaacgtgttctcttgcagcgtgatgcacgaggccctgcacaaccactacacccagaagagcctgagcctgtctccaggcaagggaggaggaagcggaggaggatctcagggacaggacaggcacatgatccggatgaggcagctgatcgacatcgtggaccagctgaagaactacgtgaacgacctggtgcccgagtttctgccagctccagaggacgtggagaccaattgcgagtggagcgccttcagttgcttccagaaggcccagctgaagagcgccaacaccggcaacaacgagcggatcatcaacgtgtccatcaagaagctgaagaggaagccccctagcacaaacgccggcagaaggcagaagcacaggctgacttgcccctcttgcgacagctacgagaagaagccccccaaggagttcctggagcggttcaagagcctgctgcagaagatgatccaccagcacctgagcagcagaacccacggaagcgaggatagc

SEQ ID NO: 14 - DNA, 642 bp, synthetic construct
  source 1..642: mol_type = other DNA
gagatcgtgatgacccagagcccagccacactgtctctgtctccaggcgagagagccacactgagttgcaaggccagccaggacgtgaagaccgccgtctcttggtaccagcagaagccaggccaggctcctaggctgctgatctattgggccagcacaagagccacaggcattccagctagattcagcggcagcggaagcggcacagattacaccctgaccatcagcagcctggagccagaggacttcgccgtctactactgccagcagcactacagcaccccttggacattcggccagggcaccaaggtggagatcaagaggaccgtggccgcccctagcgtgtttatcttccctcctagcgacgagcagctgaaaagcggaacagccagcgtcgtctgcctgctgaacaacttctaccccagggaggccaaagtccagtggaaagtggacaacgccctgcagagcggcaattctcaggagagcgtgaccgagcaggatagcaaggacagcacctacagcctgagcagcacactgaccctgagcaaggccgactacgagaagcacaaggtgtacgcttgcgaggtcacacaccagggactgagcagccccgtgaccaagagcttcaaccgcggcgagtgt

(19) United States
(12) Patent Application Publication
(10) Pub. No.: US 2026/0070977 A1
(43) Pub. Date: Mar. 12, 2026

(54) ANTI-TIGIT/RHIL-21 BIFUNCTIONAL FUSION PROTEIN AND USE THEREOF

(71) Applicant: THE FIRST AFFILIATED HOSPITAL OF NINGBO UNIVERSITY, Ningbo (CN)

(72) Inventors: Lixia Sheng, Ningbo (CN); Guifang Ouyang, Ningbo (CN); Yanli Lai, Ningbo (CN); Peng Li, Ningbo (CN)

(21) Appl. No.: 19/213,510

(22) Filed: May 20, 2025

(30) Foreign Application Priority Data
Aug. 27, 2024 (CN) 202411182134.X

(51) Int. Cl. C07K 16/28; C07K 16/24

(52) U.S. Cl. CPC C07K 16/2803; C07K 16/244; C07K 2317/24; C07K 2317/31; C07K 2317/732; C07K 2317/92

(57) ABSTRACT

An anti-TIGIT/rhIL-21 bifunctional fusion protein is provided, the bifunctional fusion protein linking rhIL-21 to an FC tail fragment of an TIGIT antibody through a linker. An Fc fragment of the anti-TIGIT/rhIL-21 bifunctional fusion protein is bonded with rhIL-21, so that in vivo stability of IL-21 is significantly enhanced, the half-life period thereof is prolonged successfully. The anti-TIGIT/rhIL-21 bifunctional fusion protein skillfully fuses an antibody for TIGIT and recombinant human interleukin-21 to effectively block immune checkpoints and efficiently activate immune cells synchronously, which not only optimizes in vivo pharmacokinetic characteristics of IL-21, but also greatly promotes mobilization and function enrichment of the immune cells.

### REFERENCE TO SEQUENCE LISTING SUBMITTED ELECTRONICALLY

The official copy of the sequence listing is submitted electronically via Patent Center as an XML formatted sequence listing with a file named NIN1-PAU57US-Sequence created on May 14, 2025 and having a size of 15,894 bytes and is filed concurrently with the specification. The sequence listing contained in this XML formatted document is part of the specification and is herein incorporated by reference in its entirety.

### BACKGROUND OF THE INVENTION

### 1. Technical Field

The present disclosure belongs to the technical field of fusion proteins, and particularly relates to an anti-TIGIT/rhIL-21 bifunctional fusion protein and use thereof.

### 2. Description of Related Art

As a current hot immune checkpoint, TIGIT is similar to DNAM-1 in structure and is competitively bound with PVR to regulate the functions of various immune cells.

T cell immunoglobulin and ITIM domain (TIGIT) is an inhibitory receptor mainly expressed on surfaces of cells such as NK cells, activated CD8+ T cells and CD4+ T cells, and Treg (regulatory T cells). TIGIT and an activated receptor DNAM-1 (CD226) both have a similar immunoglobulin-like domain and are competitively bound with PVR, but have an opposite effect on activation and function exertion of the NK cells. It may be verified through a competition experiment that TIGIT has a stronger affinity with PVR and may effectively block the bonding of DNAM-1 and PVR, thereby weakening the immunological surveillance effect of immune cells on tumor cells. After TIGIT is bound with CD155, a major inhibiting signal is started by an ITT-like sequence motif while an ITIM sequence motif mediates a smaller inhibiting signal. The ITT-like sequence motif is phosphorylated at Tyr225 and is bound with signal molecules Grb2 and β-arrestin 2 in cytoplasm to recruit SH2 phosphatase-1 (SHIP-1) containing inositol. The SHIP-1 blocks signal transduction between phosphoinositide3 kinase (PI3K) and mitogen-activated protein kinases (MAPKs) and inhibits activation of TRAF6 and NF-κB, so as to achieve negative regulation of cytokine secretion and cytotoxic activity of immune cells such as NK cells and T cells.

TIGIT has become a high-profile immune checkpoint after PD-1/PD-L1 and CTLA-4. At present, over 10 TIGIT antibodies have entered clinical research in the world and attracted the attention of massive researchers in the field of tumor immunotherapy. Clinical research and animal experiments based on solid tumors have verified that joint use of the TIGIT antibody and a PD-1 monoclonal antibody may effectively reverse immunodepletion of the T cells and NK cells in a tumor microenvironment (TME). However, an existing anti-TIGIT monoclonal antibody is less than satisfactory in terms of clinical research data in refractory and recurrent B cell lymphoma. Lymphoma TME research based on spatially resolved multi-omics technology has disclosed that TME of a significant proportion of refractory and recurrent B cell lymphoma patients is “cold tumors” with insufficient immune infiltration. Due to a shortage of sufficient immunologic effector cells, an immune checkpoint inhibitor cannot play its role effectively. The immune infiltration extent in the TME has shown a vital role in tumor progression and affects an immunotherapeutic outcome. The “cold tumors” with insufficient immune cell infiltration have a low response rate to immune checkpoint blocking agents (ICIs). Therefore, TME remodeling to increase the number of the immune cells and enhance the antitumor activity is an important strategy for improving the ICIs curative effect such as TIGIT blocking.

### BRIEF SUMMARY OF THE INVENTION

To solve the above-mentioned problem in the prior art, an anti-TIGIT/rhIL-21 bifunctional fusion protein with immune checkpoint blocking and immune cell activating functions is constructed.

An objective of the present disclosure may be achieved by the following technical solutions: An anti-TIGIT/rhIL-21 bifunctional fusion protein, linking rhIL-21 to an FC tail fragment of an TIGIT antibody through a linker.

In the anti-TIGIT/rhIL-21 bifunctional fusion protein, an amino acid sequence of the linker is as shown in SEQ ID NO: 1.

In the anti-TIGIT/rhIL-21 bifunctional fusion protein, an amino acid sequence of rhIL-21 is as shown in SEQ ID NO: 2.

In the anti-TIGIT/rhIL-21 bifunctional fusion protein, the TIGIT antibody includes a constant region sequence selected from any one of the IgG1, IgG2, IgG3, IgG4, IgA, IgM, IgE, IgD of q human or mouse antibody.”

Preferably, the TIGIT antibody is a humanized IgG1 type antibody.

In the anti-TIGIT/rhIL-21 bifunctional fusion protein, complementary determining regions (CDRs) of V_{H} of the TIGIT antibody have amino acid sequences of CDRs selected from the following group: CDR1 shown in SEQ ID NO: 3, CDR2 shown in SEQ ID NO: 4, and CDR3 shown in SEQ ID NO: 5; and

CDRs of V_{L} of the TIGIT antibody have amino acid sequences of CDRs selected from the following group: CDR1 shown in SEQ ID NO: 6, CDR2 shown in SEQ ID NO: 7, and CDR3 shown in SEQ ID NO: 8.

In the anti-TIGIT/rhIL-21 bifunctional fusion protein, amino acid sequences of V_{H} and V_{L} of the TIGIT antibody are as shown in SEQ ID NO: 9 and SEQ ID NO: 10, respectively.

The present disclosure further provides a nucleic acid molecule, encoding the TIGIT antibody.

The present disclosure further provides an expression vector, comprising the nucleic acid molecule.

The present disclosure further provides a recombinant vector, comprising the nucleic acid molecule.

The present disclosure further provides a pharmaceutical composition for treating or inhibiting tumor cell proliferation or tumor cell metastasis-related diseases or symptoms, comprising the anti-TIGIT/rhIL-21 bifunctional fusion protein.

In the pharmaceutical composition for treating or inhibiting tumor cell proliferation or tumor cell metastasis-related diseases or symptoms, the pharmaceutical composition further includes a pharmaceutically acceptable vector or accessory.

In the pharmaceutical composition for treating or inhibiting tumor cell proliferation or tumor cell metastasis-related diseases or symptoms, the vector includes at least one of a troche, a capsule, and an injection.

In the pharmaceutical composition for treating or inhibiting tumor cell proliferation or tumor cell metastasis-related diseases or symptoms, the accessory includes at least one of a filler, a bonding agent, a disintegrating agent, a coating material, and a preservative.

The present disclosure further provides a detection kit, comprising the anti-TIGIT/rhIL-21 bifunctional fusion protein.

Compared with the prior art, the present disclosure has the following beneficial effects:
1. The anti-TIGIT/rhIL-21 bifunctional fusion protein provided by the present disclosure skillfully fuses an antibody for TIGIT and recombinant human interleukin-21 (rhIL-21) to effectively block immune checkpoints and efficiently activate immune cells synchronously, which not only optimizes in vivo pharmacokinetic characteristics of IL-21, but also greatly promotes mobilization and function enrichment of the immune cells to provide an innovative strategy to solve a “cold tumor” problem and improve an immunotherapeutic effect, thereby exhibiting a broad application prospect.
2. A variable region of the anti-TIGIT/rhIL-21 bifunctional fusion protein provided by the present disclosure is bound with a TIGIT protein on the surface of the immune cell. This mechanism effectively blocks the interaction between TIGIT and PVR to interrupt a TIGIT-mediated immunosuppressive signal path and effectively reverse depletion of the immune cells in the anti-tumor process, so that the anti-cancer activity of the immune system is recovered.
3. An Fc fragment of the anti-TIGIT/rhIL-21 bifunctional fusion protein provided by the present disclosure is bound with rhIL-21, so that the in vivo stability of IL-21 is significantly enhanced, the half-life period thereof is prolonged successfully, and the limitation that the half-life period is short when rhIL-21 is independently used is overcome, thereby providing a powerful guarantee for continuous treatment.
4. In the anti-TIGIT/rhIL-21 bifunctional fusion protein provided by the present disclosure, the structure of the anti-TIGIT antibody endows the fusion protein with unique immune homing. The antigen-binding sites in the variable region may be bound with key immunologic effector cells such as NK cells and T cells that express TIGIT, and their Fc terminals may also interact with the Fc receptors (FcR) on the surfaces of other immune cells such as NK cells and macrophages, which, thus, promotes not only enrichment of IL-21 in the tumor microenvironment, but also efficient bonding of IL-21 and the IL-21 receptors (IL-21R), so as to significantly enhance proliferation and activation of these immune cells, thereby motivating a stronger antitumor immune response.

### BRIEF DESCRIPTION OF THE SEVERAL VIEWS OF THE DRAWINGS

FIG. 1 is a schematic structural diagram of a molecule of an anti-TIGIT/rhIL-21 bifunctional fusion protein prepared in Example 1.

FIGS. 2A-2E illustrate an expression vector for the anti-TIGIT/rhIL-21 bifunctional fusion protein prepared in Example 1; FIG. 2A shows amino acid sequences of an anti-TIGIT light chain, an anti-TIGIT heavy chain, and a rhIL-21 concatemer after optimization design with codon optimization software MaxCodon™ Optimization Program (V13); FIG. 2B is a schematic structural diagram of a vector of a light chain gene for synthesizing the anti-TIGIT/rhIL-21 bifunctional fusion protein by using a complete genome; FIG. 2C is a schematic structural diagram of a heavy chain gene for synthesizing the anti-TIGIT/rhIL-21 bifunctional fusion protein; FIG. 2D is a schematic diagram where a TIGIT-LC expression vector is constructed; and FIG. 2E is a schematic diagram where a TIGIT-HC expression vector is constructed.

FIG. 3 is a verification report of a light chain vector of the anti-TIGIT/rhIL-21 bifunctional fusion protein prepared in Example 1; The light chain gene is synthesized by using the complete genome and is inserted into an expression vector pcDNA3.1(−) by double digestion, and the accuracy of the constructed light chain vector is verified by digestion.

FIG. 4 is a verification report of a heavy chain vector of the anti-TIGIT/rhIL-21 bifunctional fusion protein prepared in Example 1; The heavy chain gene is synthesized by using the complete genome and is inserted into an expression vector pcDNA3.1(−) by double digestion, and the accuracy of the constructed heavy chain vector is verified by digestion.

FIG. 5 shows SDS-PAGE detection results in different links in a process of purifying the anti-TIGIT/rhIL-21 bifunctional fusion protein prepared in Example 1.

FIG. 6 shows an SDS-PAGE detection result of a product obtained by purifying the anti-TIGIT/rhIL-21 bifunctional fusion protein prepared in Example 1.

FIG. 7 is a diagram of affinity measuring parameters between the anti-TIGIT/rhIL-21 bifunctional fusion protein prepared in Example 1 and a target protein human IL-21 protein and human TIGIT protein.

FIG. 8 shows a diagram of an affinity measuring result between the anti-TIGIT/rhIL-21 bifunctional fusion protein prepared in Example 1 and the target protein human TIGIT protein.

FIG. 9 shows a diagram of an affinity measuring result between the anti-TIGIT/rhIL-21 bifunctional fusion protein prepared in Example 1 and the target protein human IL-21 protein.

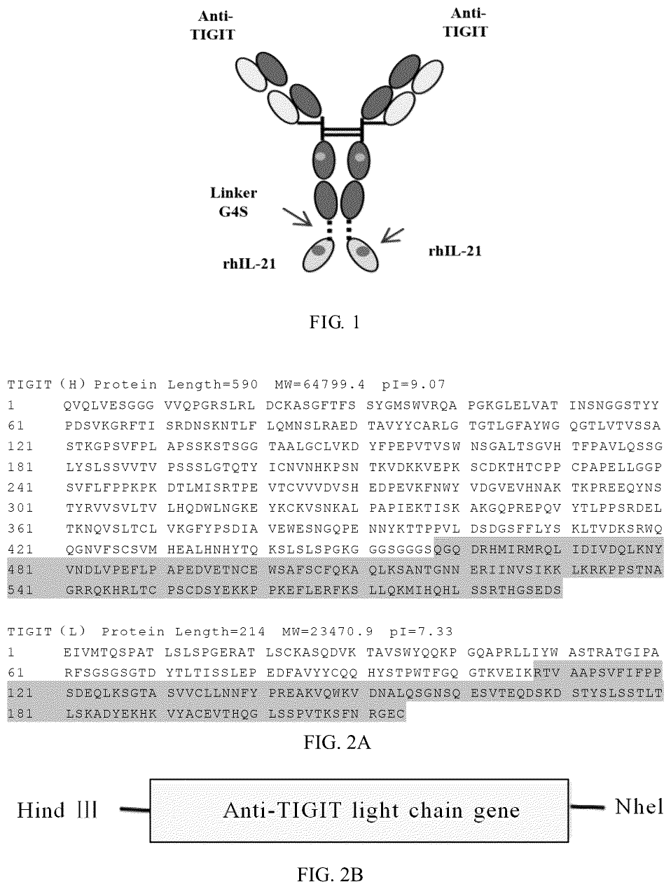

### DETAILED DESCRIPTION OF THE INVENTION

The technical solutions of the present disclosure are further described below in conjunction with specific examples and drawings. It should be understood that the specific examples described herein are merely used for describing the present disclosure, rather than limiting the scope of the present disclosure. Furthermore, the drawings used herein are merely used for better illustrating contents disclosed by the present disclosure, rather than limiting the protection scope. In addition, it should be understood that after reading the content taught in the present disclosure, those of ordinary skill may make various alterations or modifications on the present disclosure. These equivalent forms also fall within the scope defined by the appended claims of the present application.

### Example 1

### (1) Construction of a Humanized TIGIT Antibody in Genetic Engineering

A mouse-derived anti-TIGIT antibody obtained from a mouse hybridoma in a preliminary laboratory was subjected to amino acid sequencing, variable region sequences of a light chain and a heavy chain of the antibody bound with TIGIT were selected, a constant region of the antibody was humanized with mild type human IgG1 with an ADCC effect, and amino acid sequences of the anti-TIGIT were optimized by using codon optimization software MaxCodon™ Optimization Program (V13), as shown in SEQ ID NO: 9-10 to obtain a DNA sequence of the humanized TIGIT antibody in a recombinant protein.

### (2) Construction of an Expression Vector of the Anti-TIGIT/rhIL-21 Bifunctional Fusion Protein

A gene sequence of rhIL-21 encoding the amino acid sequence shown in SEQ ID NO: 2 was linked to an FC tail fragment (C terminals of two heavy chains) of the DNA sequence of the humanized TIGIT antibody by linking a linked G4S encoding the amino acid sequence shown in SEQ ID NO: 1 to the DNA sequence, complementary determining regions (CDRs) of V_{H} of the TIGIT antibody had amino acid sequences of CDRs selected from the following group: CDR1 shown in SEQ ID NO: 3, CDR2 shown in SEQ ID NO: 4, and CDR3 shown in SEQ ID NO: 5;

CDRs of V_{L} of the TIGIT antibody had amino acid sequences of CDRs selected from the following group: CDR1 shown in SEQ ID NO: 6, CDR2 shown in SEQ ID NO: 7, and CDR3 shown in SEQ ID NO: 8.

TABLE 2
Specific amino acid sequence information of the anti-TIGIT/rhIL-
21 bifunctional fusion protein

| Name | Sequence No. | Specific amino acid sequence |
|---|---|---|
| Variable region | CDR1 is shown in | GFTFSSYG |
| of heavy chain | SEQ ID NO: 3 | |
| of TIGIT/ | CDR2 is shown in | INSNGGST |
| rhIL-21 | SEQ ID NO: 4 | |
| | CDR3 is shown in | ARLGTGTLGFAY |
| | SEQ ID NO: 5 | |
| Variable region | CDR1 is shown in | QDVKTA |
| of light | SEQ ID NO: 6 | |
| chain of | CDR1 is shown in | WAS |
| TIGIT/rhIL-21 | SEQ ID NO: 7 | |
| | CDR1 is shown in | QQHYSTPWT |
| | SEQ ID NO: 8 | |
| Linker | SEQ ID NO: 1 | GGGSGGGS |
| rhIL-21 | SEQ ID NO: 2 | QGQDRHMIRMRQLIDIVDQLKNYVNDLVPEFLPAPEDVE |
| | | TNCEWSAFSCFQKAQLKSANTGNNERIINVSIKKLKRKP |
| | | PSTNAGRRQKHRLTCPSCDSYEKKPPKEFLERFKSLLQK |
| | | MIHQHLSSRTHGSEDS |
| Heavy chain of | SEQ ID NO: 9 | QVQLVESGGGVVQPGRSLRLDCKASGFTFSSYGMSWVRQ |
| TIGIT/rhIL-21 | | APGKGLELVATINSNGGSTYYPDSVKGRFTISRDNSKNT |
| | | LFLQMNSLRAEDTAVYYCARLGTGTLGFAYWGQGTLVTV |
| | | SSASTKGPSVFPLAPSSKSTSGGTAALGCLVKDYFPEPV |
| | | TVSWNSGALTSGVHTFPAVLQSSGLYSLSSVVTVPSSSL |
| | | GTQTYICNVNHKPSNTKVDKKVEPKSCDKTHTCPPCPAP |
| | | ELLGGPSVFLFPPKPKDTLMISRTPEVTCVVVDVSHEDP |
| | | EVKFNWYVDGVEVHNAKTKPREEQYNSTYRVVSVLTVLH |
| | | QDWLNGKEYKCKVSNKALPAPIEKTISKAKGQPREPQVY |
| | | TLPPSRDELTKNQVSLTCLVKGFYPSDIAVEWESNGQPE |
| | | NNYKTTPPVLDSDGSFFLYSKLTVDKSRWQQGNVFSCSV |
| | | MHEALHNHYTQKSLSLSPGKGGGSGGGSQGQDRHMIRMR |
| | | QLIDIVDQLKNYVNDLVPEFLPAPEDVETNCEWSAFSCF |
| | | QKAQLKSANTGNNERIINVSIKKLKRKPPSTNAGRRQKH |
| | | RLTCPSCDSYEKKPPKEFLERFKSLLQKMIHQHLSSRTH |
| | | GSEDS |
| Light chain of | SEQ ID NO: 10 | EIVMTQSPATLSLSPGERATLSCKASQDVKTAVSWYQQK |
| TIGIT/rhIL-21 | | PGQAPRLLIYWASTRATGIPARFSGSGSGTDYTLTISSL |
| | | EPEDFAVYYCQQHYSTPWTFGQGTKVEIKRTVAAPSVFI |
| | | FPPSDEQLKSGTASVVCLLNNFYPREAKVQWKVDNALQS |
| | | GNSQESVTEQDSKDSTYSLSSTLTLSKADYEKHKVYACE |
| | | VTHQGLSSPVTKSFNRGEC |

TABLE 3
Specific DNA sequence information of the anti-TIGIT/rhIL-21
bifunctional fusion protein

| Name | Sequence No. | Specific DNA sequence |
|---|---|---|
| Linker | SEQ ID NO: 11 | GGCGGCGGCGGCAGC |
| rhIL-21 | SEQ ID NO: 12 | CAGGGCCAGGACAGGCACATGATCAGGATGAGGCA |
| | | GCTGATCGACATCGTGGACCAGCTGAAGAACTACG |
| | | TGAACGACCTGGTGCCCGAGTTCCTGCCCGCCCCC |
| | | GAGGACGTGGAGACCAACTGCGAGTGGAGCGCCTT |
| | | CAGCTGCTTCCAGAAGGCCCAGCTGAAGAGCGCCA |
| | | ACACCGGCAACAACGAGAGGATCATCAACGTGAGC |
| | | ATCAAGAAGCTGAAGAGGAAGCCCCCCAGCACCAA |
| | | CGCCGGCAGGAGGCAGAAGCACAGGCTGACCTGCC |
| | | CCAGCTGCGACAGCTACGAGAAGAAGCCCCCCAAG |
| | | GAGTTCCTGGAGAGGTTCAAGAGCCTGCTGCAGAA |
| | | GATGATCCACCAGCACCTGAGCAGCAGGACCCACG |
| | | GCAGCGAGGACAGC |
| Heavy chain of | SEQ ID NO: 13 | CAGGTGCAGCTGGTGGAATCAGGAGGAGGAGTGGT |
| TIGIT/rhIL-21 | | GCAGCCAGGAAGAAGCCTGAGACTGGATTGCAAGG |
| | | CCAGCGGCTTCACCTTCAGCAGCTACGGAATGTCT |
| | | TGGGTCCGGCAGGCTCCAGGAAAAGGACTGGAACT |
| | | GGTGGCCACCATCAACAGCAACGGCGGCAGCACCT |
| | | ACTACCCAGACAGCGTGAAGGGCAGGTTCACCATC |
| | | AGCCGGGACAACAGCAAGAACACCCTGTTCCTGCA |
| | | GATGAACAGCCTGAGGGCCGAGGATACCGCCGTGT |
| | | ACTATTGCGCTAGACTGGGCACCGGCACACTGGGA |
| | | TTTGCCTATTGGGGCCAGGGCACACTGGTGACAGT |
| | | GTCTAGCGCCAGCACCAAGGGCCCTAGCGTGTTTC |
| | | CTCTGGCCCCTTCTAGCAAGTCTACAAGCGGCGGA |
| | | ACAGCCGCTCTGGGTTGCCTGGTGAAAGACTACTT |
| | | CCCCGAGCCAGTGACCGTGTCTTGGAATAGCGGAG |
| | | CCCTGACCAGCGGAGTGCATACCTTTCCAGCCGTG |
| | | CTGCAGAGCTCAGGACTGTACAGCCTGTCCAGCGT |
| | | GGTGACAGTGCCTTCTAGCAGCCTGGGCACACAGA |
| | | CCTACATCTGCAACGTGAACCACAAGCCCAGCAAC |
| | | ACCAAGGTGGACAAGAAGGTGGAGCCCAAGAGCTG |
| | | CGACAAGACCCACACTTGCCCTCCTTGTCCAGCTC |
| | | CAGAACTGCTGGGAGGACCAAGCGTGTTCCTGTTC |
| | | CCTCCCAAGCCCAAGGACACCCTGATGATCAGCAG |
| | | GACCCCCGAAGTGACTTGCGTGGTGGTGGACGTGT |
| | | CTCACGAGGACCCCGAGGTCAAGTTCAATTGGTAC |
| | | GTGGACGGAGTGGAGGTGCACAACGCTAAGACCAA |
| | | GCCCAGGGAGGAGCAGTACAACAGCACCTACAGGG |
| | | TGGTGTCCGTGCTGACAGTGCTGCACCAGGATTGG |
| | | CTGAACGGCAAGGAGTACAAGTGCAAGGTGTCCAA |
| | | CAAGGCTCTGCCAGCCCCCATCGAGAAGACCATCA |
| | | GCAAGGCTAAGGGCCAGCCTAGAGAACCTCAGGTG |
| | | TACACCCTGCCCCCTTCTAGAGACGAGCTGACCAA |
| | | GAACCAGGTGTCCCTGACTTGCCTCGTGAAGGGCT |
| | | TCTACCCCAGCGATATCGCCGTGGAGTGGGAATCT |
| | | AACGGCCAGCCAGAGAACAACTACAAGACCACCCC |
| | | CCCAGTGCTGGACAGCGACGGCAGCTTCTTCCTGT |
| | | ACAGCAAGCTGACCGTGGACAAAAGCCGCTGGCAG |
| | | CAGGGCAACGTGTTCTCTTGCAGCGTGATGCACGA |
| | | GGCCCTGCACAACCACTACACCCAGAAGAGCCTGA |
| | | GCCTGTCTCCAGGCAAGGGAGGAGGAAGCGGAGGA |
| | | GGATCTCAGGGACAGGACAGGCACATGATCCGGAT |
| | | GAGGCAGCTGATCGACATCGTGGACCAGCTGAAGA |
| | | ACTACGTGAACGACCTGGTGCCCGAGTTTCTGCCA |
| | | GCTCCAGAGGACGTGGAGACCAATTGCGAGTGGAG |
| | | CGCCTTCAGTTGCTTCCAGAAGGCCCAGCTGAAGA |
| | | GCGCCAACACCGGCAACAACGAGCGGATCATCAAC |
| | | GTGTCCATCAAGAAGCTGAAGAGGAAGCCCCCTAG |
| | | CACAAACGCCGGCAGAAGGCAGAAGCACAGGCTGA |
| | | CTTGCCCCTCTTGCGACAGCTACGAGAAGAAGCCC |
| | | CCCAAGGAGTTCCTGGAGCGGTTCAAGAGCCTGCT |
| | | GCAGAAGATGATCCACCAGCACCTGAGCAGCAGAA |
| | | CCCACGGAAGCGAGGATAGCGAGATCGTGATGACC |
| | | CAGAGCCCAGCCACACTGTCTCTGTCTCCAGGCGA |
| | | GAGAGCCACACTGAGTTGCAAGGCCAGCCAGGACG |
| | | TGAAGACCGCCGTCTCTTGGTACCAG |
| Light chain of | SEQ ID NO: 14 | CAGAAGCCAGGCCAGGCTCCTAGGCTGCTGATCTA |
| TIGIT/rhIL-21 | | TTGGGCCAGCACAAGAGCCACAGGCATTCCAGCTA |
| | | GATTCAGCGGCAGCGGAAGCGGCACAGATTACACC |
| | | CTGACCATCAGCAGCCTGGAGCCAGAGGACTTCGC |
| | | CGTCTACTACTGCCAGCAGCACTACAGCACCCCTT |
| | | GGACATTCGGCCAGGGCACCAAGGTGGAGATCAAG |
| | | AGGACCGTGGCCGCCCCTAGCGTGTTTATCTTCCC |
| | | TCCTAGCGACGAGCAGCTGAAAAGCGGAACAGCCA |
| | | GCGTCGTCTGCCTGCTGAACAACTTCTACCCCAGG |
| | | GAGGCCAAAGTCCAGTGGAAAGTGGACAACGCCCT |
| | | GCAGAGCGGCAATTCTCAGGAGAGCGTGACCGAGC |
| | | AGGATAGCAAGGACAGCACCTACAGCCTGAGCAGC |
| | | ACACTGACCCTGAGCAAGGCCGACTACGAGAAGCA |
| | | CAAGGTGTACGCTTGCGAGGTCACACACCAGGGAC |
| | | TGAGCAGCCCCGTGACCAAGAGCTTCAACCGCGGC |
| | | GAGTGT |

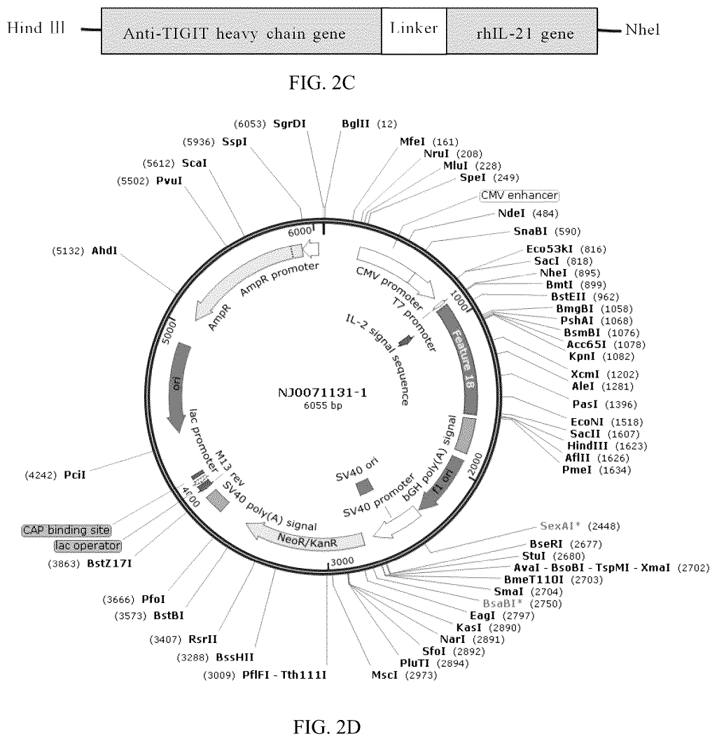

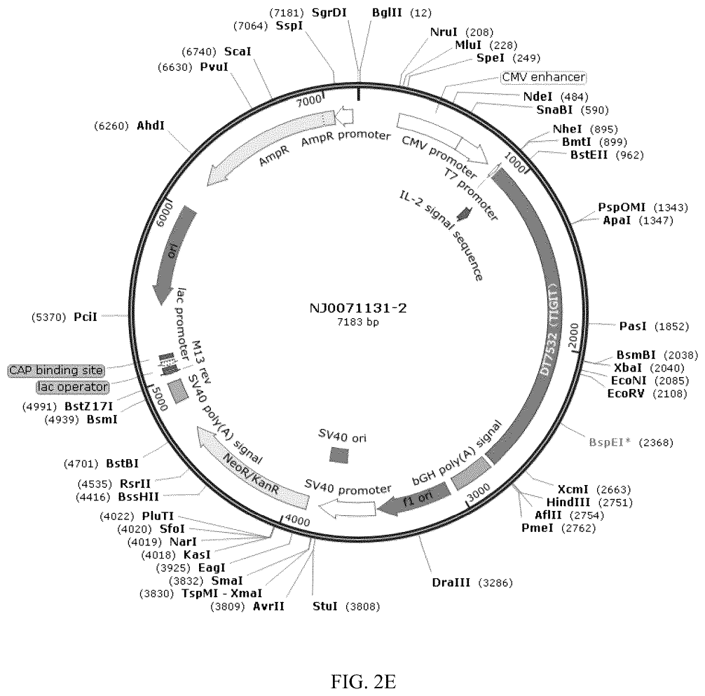

A light chain gene sequence of the bifunctional fusion protein and a heavy chain gene sequence of the bifunctional fusion protein were synthesized by using a complete genome, a light chain gene and a heavy chain gene of the anti-TIGIT/rhIL-21 fusion protein were inserted into an expression vector pcDNA3.1 by double digestion, respectively to construct two expression vectors: pcDNA3.1-DT7532 (TIGIT-LC) (FIG. 2D) and pcDNA3.1-DT7532 (TIGIT-HC) (FIG. 2E), and the accuracy of the final expression vector was determined by digestion and sequencing.

FIG. 2 is a construction diagram of an expression vector for the anti-TIGIT/rhIL-21 bifunctional fusion protein; FIG. 2B is a schematic structural diagram of a light chain gene of the anti-TIGIT/rhIL-21 bifunctional fusion protein synthesized by using a complete genome; FIG. 2C is a schematic structural diagram of a heavy chain gene for synthesizing the anti-TIGIT/rhIL-21 bifunctional fusion protein; FIG. 2D is a schematic diagram where an expression vector of the pcDNA3.1-DT7532 (TIGIT-LC) light chain gene is constructed; and FIG. 2E is a schematic diagram where an expression vector of the pcDNA3.1-DT7532 (TIGIT-HC) heavy chain gene is constructed.

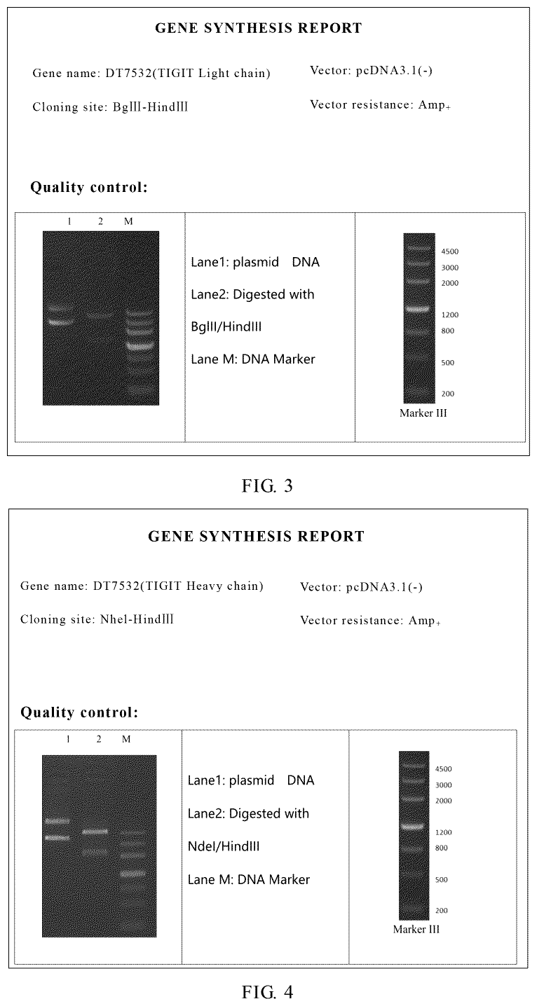

FIG. 3 is a verification report of a light chain vector of the anti-TIGIT/rhIL-21 bifunctional fusion protein. It could be known from FIG. 3 that the expression vector of the light chain gene of the anti-TIGIT/rhIL-21 bifunctional fusion protein was successfully synthesized. A target sequence length of a DT7532-1 light chain is 734 bp, a restriction enzyme cutting site is BglII/HindIII, and double digestion strips are respectively located at 1611 bp and 4444 bp.

FIG. 4 is a verification report of a heavy chain vector of the anti-TIGIT/rhIL-21 bifunctional fusion protein. It could be known from FIG. 4 that the expression vector of the heavy chain gene of the anti-TIGIT/rhIL-21 bifunctional fusion protein was successfully synthesized. A target sequence length of a DT7532-1 heavy chain is 1862 bp, a restriction enzyme cutting site is BglII/HindIII, and double digestion strips are respectively located at 2267 bp and 4916 bp.

### (3) Transfection Level Plasmid Amplification and Extraction

80-100 ng of two expression plasmids pcDNA3.1-DT7532 (TIGIT-LC) and pcDNA3.1-DT7532 (TIGIT-HC) was sucked and respectively added into prepared DH5a competent cells, the competent cells with the plasmids were converted by heat shock, the converted competent cells were added into an LB liquid culture medium, the LB liquid culture medium was placed in a table for shake culture at 37° C. and 200 rpm for 30 min; the cultured competent cells were taken out, a part of suspension was sucked and coated to a plate containing ampicillin resistance, and the plate was placed in an incubator for overnight culture at 37° C.; a monoclone was picked from a fresh culture plate and placed in a 2-5 mL LB culture medium for culture at 37° C. and 200 rpm for 8 h; the mixture was inoculated in a proportion of 1/500 to a 200 mL LB culture medium for culture at 37° C. and 200 rpm for 16 h; a bacteria solution after culture was collected and centrifuged, and a supernatant was removed; and plasmids were extracted by using a Qiagen transfection level plasmid extraction kit, and the extracted plasmids were electrophoretically analyzed by a 1% agarose gel.

### (4) Transfection of a Vector Carrying Two Target Genes to HEK293 Cells

The HEK293 cells were subjected to suspension culture to 1 L 1 day before transfection, with an inoculating density of 1×10⁶ cells/mL, and the cells were placed in an incubator for culture at 110 rpm and 37° C. with 5% CO₂. The cell density on the day of transfection was adjusted to about 1-1.5×10⁶ cells/mL. The plasmids and a transfection reagent were added into a transfection buffer to be uniformly mixed, a DNA-transfection reagent mixture was added into to-be-transfected cells, and the to-be-transfected cells were placed in the incubator for culture at 110 rpm and 37° C. with 5% CO₂. In about 4-6 days after transfection, a cell culture was taken out and centrifuged to collect the supernatant.

### (5) Purification of the Bifunctional Fusion Protein

A cell culture solution transfected for 5 days was centrifuged, the supernatant was filtered with a 0.22 μm filter, dialyzed to 1×PBS of the buffer with pH 7.4 in an environment at 4° C., purified by a proteinA column after dialysis, purified by Ni-IDA affinity chromatography, and then purified by SP Beads 6FF ion-exchange column chromatography. Lane 3 with relatively high purity was collected, dialyzed to 1×PBS of the buffer with pH 7.4, filtered with the 0.22 μm filter after dialysis, and divided into two parts: one part was subpackaged for freeze-drying and the other part was cryopreserved to −80° C.

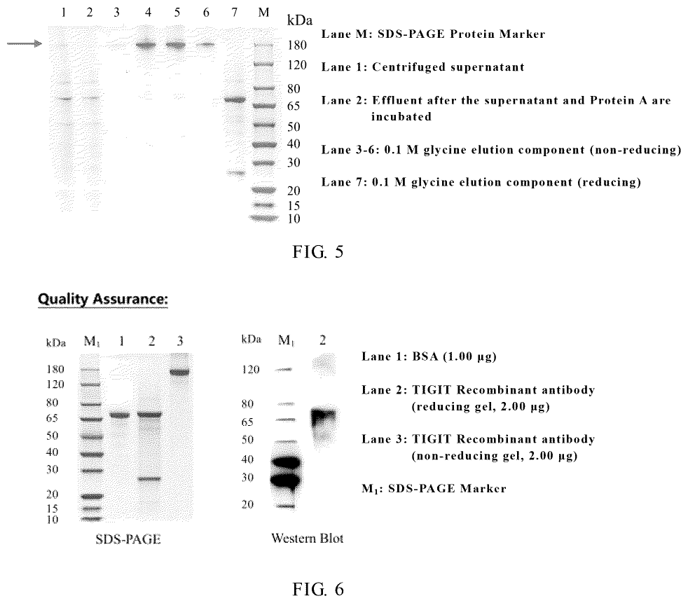

FIG. 5 is a control diagram of a purification process of the anti-TIGIT/rhIL-21 bifunctional fusion protein. As demonstrated in FIG. 5 that the lane1 strip means that the concentration of the target protein is low in the cultural supernatant before purification, and the contents of other non-target proteins are high; the lane2 strip means that in an effluent bound with the recombinant protein by the proteinA column, there is no target protein strip; lanes 3-6 mean that the strips of the recombinant strip eluted by glycine in a non-reducing state are clear, with the molecular weight about 180 KDa; and lane7 means that there are two strips of the recombinant strip eluted by glycine in the non-reducing state, respectively representing the heavy chain (molecular weight about 65 KDa) and the light chain (molecular weight about 25 KDa).

### (6) Identification of the Bifunctional Fusion Protein

The bifunctional fusion protein was obtained after purification of the cultural supernatant and replacement of the buffer, and the molecular weight and the structural integrity of the bifunctional fusion protein were identified by using reducing and non-reducing SDS-PAGE.

FIG. 6 is a diagram of purification of the anti-TIGIT/rhIL-21 bifunctional fusion protein analyzed by SDS-PAGE. As demonstrated in “FIG. 6 that in the lane3 of non-reducing SDS-PAGE, the strip of the recombinant protein with an integral structure is located near 180 KDa; and in the lane2 of reducing SDS-PAGE, strips of the light chain and the heavy chain of the recombinant protein are respectively located near 25 KDa and 65 KDa.

### (7) Measurement of an Affinity Between the Bifunctional Fusion Protein and the Target Protein TIGIT

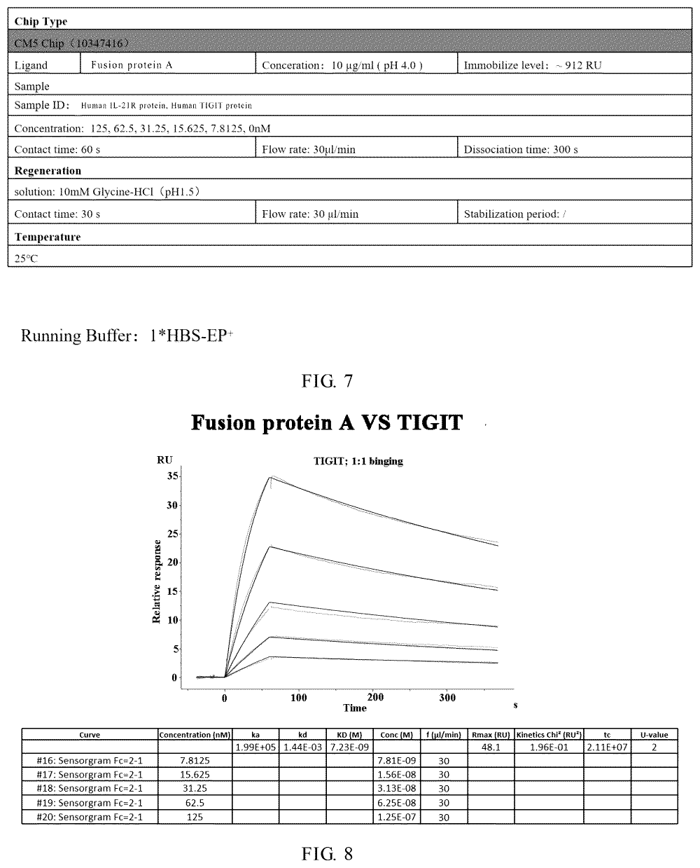

As demonstrated in FIGS. 7-8 that the affinity KD value between the anti-TIGIT/rhIL-21 bifunctional fusion protein and the human TIGIT protein measured by surface plasmon resonance (SPR) is 7.23E-9 (M), indicating a high affinity with the target protein.

### (8) Measurement of an Affinity Between the Bifunctional Fusion Protein and the Target Protein Human IL-21R:

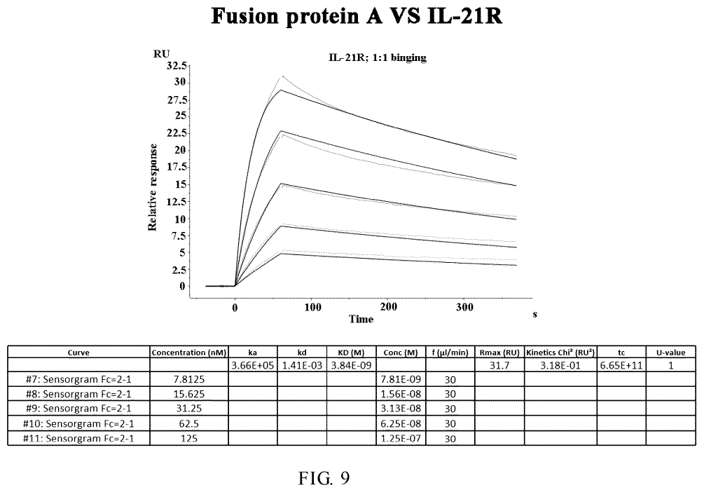

FIG. 9 is a diagram of an affinity measuring result between the anti-TIGIT/rhIL-21 bifunctional fusion protein and the target protein human IL-21 protein. As demonstrated in FIG. 9 that the affinity KD value between the recombinant fusion protein and the human IL-21 receptor protein (IL-21R) measured by SPR is 3.84E-9 (M), indicating a high affinity with the target protein.

All aspects, examples, and features of the present disclosure should be regarded as being illustrative in all aspects rather than limiting the present disclosure. The scope of the present disclosure is only defined by the claims. Those skilled in the art will clearly understand other examples, modifications, and use without departing from the spirit and scope of the claimed present disclosure.

In the preparation method of the present disclosure, the order of the steps is not limited to the order listed. For those of ordinary skill in the art, successive changes of the steps also fall within the protection scope of the present disclosure without creative labor. In addition, two or more than two steps or actions may be performed simultaneously.

It should be noted finally that the specific examples described herein are merely illustrations of the present disclosure, rather than limiting the implementation of the present disclosure. Those skilled in the art may make various modifications or supplementations on the specific examples described or replace the modifications or supplementations in a similar manner, and it is neither necessary nor possible to provide a complete example for all implementations here. These apparent changes or alternations derived from the essential spirit of the present disclosure still fall within the protection scope of the present disclosure, and explanation of the changes or alternations as any additional limit is contrary to the spirit of the present disclosure.

What is claimed is:

1. An anti-TIGIT/rhIL-21 bifunctional fusion protein, linking rhIL-21 to an FC tail fragment of an TIGIT antibody through a linker.

2. The anti-TIGIT/rhIL-21 bifunctional fusion protein according to claim 1, wherein an amino acid sequence of the linker is as shown in SEQ ID NO: 1.

3. The anti-TIGIT/rhIL-21 bifunctional fusion protein according to claim 1, wherein an amino acid sequence of rhIL-21 is as shown in SEQ ID NO: 2.

4. The anti-TIGIT/rhIL-21 bifunctional fusion protein according to claim 1, wherein the TIGIT antibody comprises a constant region sequence selected from any one of the IgG1, IgG2, IgG3, IgG4, IgA, IgM, IgE, IgD of q human or mouse antibody.

5. The anti-TIGIT/rhIL-21 bifunctional fusion protein according to claim 1, wherein the TIGIT antibody is a humanized IgG1 type antibody.

6. The anti-TIGIT/rhIL-21 bifunctional fusion protein according to claim 1, wherein complementary determining region CDRs of V_{H} of the TIGIT antibody have amino acid sequences of CDRs selected from the following group: CDR1 shown in SEQ ID NO: 3, CDR2 shown in SEQ ID NO: 4, and CDR3 shown in SEQ ID NO: 5; and
CDRs of V_{L} of the TIGIT antibody have amino acid sequences of CDRs selected from the following group: CDR1 shown in SEQ ID NO: 6, CDR2 shown in SEQ ID NO: 7, and CDR3 shown in SEQ ID NO: 8.

7. The anti-TIGIT/rhIL-21 bifunctional fusion protein according to claim 1, wherein amino acid sequences of V_{H} and V_{L} of the TIGIT antibody are as shown in SEQ ID NO: 9 and SEQ ID NO: 10, respectively.

8. A nucleic acid molecule, encoding the TIGIT antibody according to claim 1.

9. An expression vector, comprising the nucleic acid molecule according to claim 8.

10. A recombinant vector, comprising the nucleic acid molecule according to claim 8.

11. A pharmaceutical composition for treating or inhibiting tumor cell proliferation or tumor cell metastasis-related diseases or symptoms, comprising the anti-TIGIT/rhIL-21 bifunctional fusion protein according to claim 1.

12. The pharmaceutical composition for treating or inhibiting tumor cell proliferation or tumor cell metastasis-related diseases or symptoms according to claim 11, further comprising a pharmaceutically acceptable vector or accessory.

13. The pharmaceutical composition for treating or inhibiting tumor cell proliferation or tumor cell metastasis-related diseases or symptoms according to claim 12, wherein the vector comprises at least one of a troche, a capsule, and an injection.

14. The pharmaceutical composition for treating or inhibiting tumor cell proliferation or tumor cell metastasis-related diseases or symptoms according to claim 12, wherein the accessory comprises at least one of a filler, a bonding agent, a disintegrating agent, a coating material, and a preservative.

15. A detection kit, comprising the anti-TIGIT/rhIL-21 bifunctional fusion protein according to claim 1.
